(19) United States
(12) Patent Application Publication
(10) Pub. No.: US 2015/0269724 A1
(43) Pub. Date: Sep. 24, 2015

(54) DIGITAL IMAGE PROCESSING METHOD AND IMAGING APPARATUS

(71) Applicant: SHIMADZU CORPORATION, Kyoto-shi, Kyoto (JP)

(72) Inventor: Tetsuya Kobayashi, Kyoto (JP)

(73) Assignee: SHIMADZU CORPORATION, Kyoto (JP)

(21) Appl. No.: 14/431,416

(22) PCT Filed: Jun. 16, 2013
(86) PCT No.: PCT/JP2013/069283

§ 371 Date: Mar. 26, 2015

(30) Foreign Application Priority Data
Sep. 28, 2012 (JP) PCT/JP2012/006248

(51) Int. Cl. G06T 7/00; A61B 6/03; A61B 5/055; G06T 5/00; G06T 5/20

(52) U.S. Cl. CPC G06T 7/0012; G06T 5/003; G06T 5/20; A61B 5/055; A61B 6/032; A61B 6/037; G06T 2207/10104; G06T 2207/10081; G06T 2207/10088; G06T 2207/10108; G06T 2207/20028; G06T 2207/20192; G06T 2207/30004

(57) ABSTRACT

In a digital image processing, a weighting function having a pixel-to-pixel distance as a variable and a weighting function having a pixel-value difference of a neighboring pixel as a variable are each set (Step S4) for filtering with a bilateral filter. At this time, the weighting function applying weight depending on edge strength of a CT image (pixel-value difference between an adjacent pixel and a target pixel) is set using the CT image as another digital image without a PET image to be processed. As noted above, a filter coefficient is determined also with the CT image as the other digital image (Step S6). Then filtering is performed to the PET image as a digital image with the determined filter coefficient (Step S7). Consequently, filtering is obtainable with no influence of a noise level of the PET image, achieving maintenance of a space resolution and noise reduction.

### TECHNICAL FIELD

The present invention relates to a digital image processing method for processing a digital image, and an imaging apparatus that performs imaging. More particularly, the present invention is directed to a technique to determine a filter coefficient in accordance with information on a pixel-to-pixel distance and information on a pixel-value difference between a target pixel to be processed and a neighboring pixel around the target pixel.

### BACKGROUND ART

Such a digital image processing method and an imaging apparatus are used for a general medical image apparatus (e.g., a CT (Computed Tomography) apparatus, an MRI (Magnetic Resonance Imaging) apparatus, an ultrasonic tomography apparatus, and a tomography apparatus for nuclear medicine), a nondestructive testing CT apparatus, a digital camera, and a digital video camera.

One paper suggests a bilateral filter as one example of an edge-preserved smoothing filter. See, for example, Non-Patent Literature 1. Another paper describes application results of a bilateral filter to a PET (Positron Emission Tomography) image. See, for example, Non-Patent Literature 2. A weighted average filter (e.g., an average value filter and a Gaussian filter) known as the smoothing filter is used for determining a filter kernel coefficient (hereinafter, abbreviated to a “filter coefficient”) in accordance with information on a pixel-to-pixel distance between a target pixel to be processed and a neighboring pixel around the target pixel. The bilateral filter is used for determining a filter coefficient W from the following Equations (1) and (2) in accordance with information on a pixel-to-pixel distance and information on a pixel-value difference between a target pixel and a neighboring pixel.

$$\begin{matrix}W ( i , j ) = \frac{w ( i , j )}{{\sum }_{k ∈ {Ω}_{i}} w ( i , k )} & ( 1 ) \\ w ( i , j ) = {G}_{{σ}_{r}} ( ‖ r ( i ) - r ( j ) ‖ ) \times {G}_{{σ}_{x}} ( \vert x ( i ) - x ( j ) \vert ) & ( 2 )\end{matrix}$$

where i denotes an index of the target pixel, j denotes an index of neighboring pixels (adjacent pixels) to the target pixel i, w denotes a weighting factor of the neighboring pixel (adjacent pixel) j relative to the target pixel i, Ωᵢ denotes a neighboring-pixel set around the target pixel i (see FIG. 5), k denotes a variable belonging to the neighboring-pixel set Ωᵢ, r(i) denotes a position vector of the target pixel i from a reference position, r(j) denotes a position vector of the neighboring pixel j from the reference position, x(i) denotes a pixel value of the target pixel i, x(j) denotes a pixel value of the neighboring pixel (adjacent pixel) j, and G_{σ} denotes a Gaussian function of a standard deviation σ. An extent of the smoothing is determined from parameters σᵣ, σₓ (hereinafter, referred to as “smoothing parameters”) that are set depending on a characteristic of an image to be processed. As in the above Equation (2), the bilateral filter has a characteristic to preserve an edge (a pixel-value difference) in the image for decreasing a filter coefficient of paired pixels with a large pixel-value difference.

[Non-Patent Literature 1] Carlo Tomasi, Roberto Manduchi, “Bilateral Filtering for Gray and Color Images”, Proceedings of the ICCV 1998.

[Non-Patent Literature 2] Frank Hofheinz, Jens Langner, Bettina Beuthien-Baumann et al., “Suitability of bilateral filtering for edge-preserving noise reduction in PET”, EJNMMI Research, vol. 1, no. 23, 2011.

### SUMMARY OF INVENTION

### Technical Problem

However, the bilateral filter in the Background Art mentioned above possesses the following drawback. That is, the currently-used bilateral filter is likely to preserve paired pixels with a large pixel-value difference in the image to be processed as a “true edge (signal)”. However, a nuclear medicine image as representative of a PET image and a SPECT (Single Photon Emission CT) image contains large systematic and statistical variations of pixel values (hereinafter, collectively referred to as “noise”). Accordingly, a “false edge” derived from the noise is likely to be preserved as a “true edge”. Consequently, application of the bilateral filter to the nuclear medicine image causes erroneous detection of the false edge as the true edge in the nuclear medicine image.

In this case, when values of smoothing parameters σᵣ, σₓ are decreased for obtaining a maintained space resolution of the image, the false edge derived from the noise is also preserved. This causes difficulty in sufficiently eliminating the noise. In contrast to this, when values of the smoothing parameter σᵣ, σₓ are increased for enhancing noise elimination performance, the true edge also blurs. This causes difficulty in maintaining the space resolution of the image. As noted above, the currently-used technique has such a drawback that maintenance of the space resolution and noise reduction is incompatible with each other in the image to be processed with high noise.

The present invention has been made regarding the state of the art noted above, and its object is to provide a digital image processing method and an imaging apparatus that allow maintenance of a space resolution as well as noise reduction.

### Summary of Invention

The present invention is configured as under to achieve the above object. One embodiment of the present invention discloses a digital image processing method for determining a filter coefficient in accordance with information on a pixel-to-pixel distance and information on a pixel-value difference between a target pixel to be processed and a neighboring pixel around the target pixel, and processing a digital image with the determined filter coefficient. When a digital image to be processed is denoted by A and another digital image B of the same object as the digital image A is denoted by B, the digital image processing method includes determining the filter coefficient to process the digital image A also with information on the other digital image B.

With the digital image processing method according to the embodiment of the present invention, the filter coefficient is determined also with the other digital image B. This achieves filtering with no influence of a noise level of the digital image A to be processed. As a result, maintenance of the space resolution and noise reduction is both obtainable.

The other digital image B is preferably a shape image. Especially, when the image to be processed is a digital image based on nuclear medicine data (nuclear medicine image), the nuclear medicine image has physiological information, and thus is referred to as a “functional image”. On the other hand, the nuclear medicine image lacks anatomical information. Then, the shape image having anatomical information is used for the other digital image B. That is, a shape image with a high space resolution and low noise is used. This produces a more sufficient effect.

Moreover, a function having a pixel-value difference as a variable for determining the filter coefficient is preferably a non-increasing function. When the pixel-value difference is small, smoothing is performable with a function having a large value. When the pixel-value difference is large, an edge with the large difference can be preserved with a function having a small value. Here, the “non-increasing function” is applicable as long as the function does not increase with increase of the pixel-value difference. Accordingly, a value of the function may be constant at a part of the pixel-value difference. Consequently, as illustrated in FIG. 6, a part of the pixel-value difference equal to or less than a threshold (Tₐ in FIG. 6) has a constant value “a” (where a>0) (a=1 in FIG. 6), whereas a part of the pixel-value difference more than the threshold (Tₐ in FIG. 6) has a constant function “0”. A function composed of such constant values is a non-increasing function.

One example of the digital image processing method according to the present embodiment is to determine the filter coefficient from the following equation to process the digital image A to be processed. That is, in the digital image processing method according to the embodiment of the present invention, when an index of the target pixel is denoted by i, an index of the neighboring pixel relative to the target pixel i is denoted by j, a weighting factor of the neighboring pixel j relative to the target pixel i is denoted by w, a neighboring-pixel set around the target pixel i is denoted by Ωᵢ, a variable belonging to the neighboring-pixel set Ωᵢ is denoted by k, a position vector of the target pixel i from a reference position is denoted by r(i), a position vector of the neighboring pixel j from the reference position is denoted by r(j), a pixel value of a target pixel i in the other digital image B is denoted by I_{b}(i), a pixel value of the neighboring pixel j in the other digital image B is denoted by I_{b}(j), an any given function having a pixel-to-pixel distance as a variable is denoted by F, and an any given function having a pixel-value difference from the neighboring pixel in the other digital image B as a variable is denoted by H, the filter coefficient W(i,j) of the digital image A to be processed for filtering is given by equations:

W(i,j)=w(i,j)/Σw(i,k),

where Σw(i,k) is a total sum of w(i,k) of the variable k belonging to the neighboring-pixel set Ωᵢ, and

w(i,j)=F(∥r(i)−r(j)∥)×H(|I_{b}(i)−I_{b}(j)|).

From the above equation, a weighting factor w(i,j) of the neighboring pixel j relative to the target pixel i is determined with the any given function H having the pixel-value difference from the neighboring pixel in the other digital image B as the variable, and additionally the filter coefficient W(i,j) is determined with the weighting factor w(i,j). Consequently, the filter coefficient W(i,j) is determined also with the other digital image B.

Another embodiment of the present invention discloses an imaging apparatus. The imaging apparatus includes a filter determining device determining a filter coefficient in filtering, and a digital image processor processing a digital image in accordance with a captured image. When a digital image to be processed is denoted by A and another digital image B of the same object as the digital image A is denoted by B, the filter determining device determines the filter coefficient with information on a pixel-to-pixel distance and information on a pixel-value difference between a target pixel to be processed and a neighboring pixel around the target pixel and also with information on the other digital image B. The digital image processor processes the digital image A to be processed with the filter coefficient determined by the filter determining device.

The imaging apparatus according to the embodiment of the present invention includes the filter determining device determining the filter coefficient in the filtering, and the digital image processor processing the digital image based on the captured image. The filter determining device determines the filter coefficient with information on the pixel-to-pixel distance and information on the pixel-value difference between the target pixel to be processed and the neighboring pixel and also with the information on the other digital image B. The digital image processor processes the digital image A to be processed with the filter coefficient determined by the filter determining device. As noted above, the filter coefficient is determined also with the other digital image B. This achieves filtering with no influence of a noise level in the digital image A to be processed. Consequently, as described in the digital image processing method according to the present embodiment, maintenance of the space resolution and noise reduction is both obtainable.

The imaging apparatus according to the embodiment of the present invention further includes an imaging unit with a camera function for imaging a static image or a video function for imaging a moving video picture, and a digital image converting device converting the image captured by the imaging unit into a digital image. Such is preferable. With the imaging unit and the digital image converting device, the imaging unit allows imaging of the static image or the moving video picture while the digital image converting device allows conversion of the image (analog image) by the imaging unit into the digital image, and the digital image processor allows processing of the converted digital image.

As mentioned above, one example of the imaging apparatus according to the present embodiment is a nuclear medicine diagnosis apparatus conducting nuclear medicine diagnosis. The digital image processor processes a digital image in accordance with nuclear medicine data obtained from the nuclear medicine diagnosis. Such is preferable. As also described in the digital image processing method according to the present embodiment, the digital image (nuclear medicine image) based on the nuclear medicine data obtained through the nuclear medicine diagnosis is a function image, and thus lacks anatomical information. Accordingly, it is assumed that the digital image A to be processed corresponds to a digital image in accordance with the nuclear medicine data, and the other digital image B corresponds to a shape image. Consequently, use of the shape image with anatomical information as the other digital image B leads to use of a shape image with a high space resolution and low noise. As a result, maintenance of the space resolution as noise reduction is both obtainable even when a digital image to be processed is a nuclear medicine image as a function image lacking anatomical information.

With the digital image processing method and the imaging apparatus according to the present embodiments, the filter coefficient is determined also with the other digital image B. This achieves the filtering with no influence of a noise level in the digital image A to be processed. Consequently, maintenance of the space resolution and noise reduction is both obtainable.

### BRIEF DESCRIPTION OF DRAWINGS

FIG. 1 is a side view of a PET-CT apparatus according to one embodiment of the present invention.

FIG. 2 is a block diagram of the PET-CT apparatus according to the embodiment.

FIG. 3 schematically illustrates a concrete construction of a γ-ray detector.

FIG. 4 is a flow chart illustrating a series of digital image processing containing filtering.

FIG. 5 schematically illustrates a neighboring-pixel set.

FIG. 6 illustrates one example of a non-increasing function applying a weighting factor and having a pixel-value difference of neighboring pixels as a variable.

FIG. 7 is a schematic view of a filter kernel.

FIG. 8 schematically illustrates a neighboring-pixel set according to one modification of the present invention.

FIG. 9 is an original image used for demonstration data (simulation experiments).

FIG. 10 illustrates an image with noise obtained by artifactually applying noises to an original image of FIG. 9.

FIG. 11 is a shape image used for demonstration data (simulation experiments).

FIGS. 12 to 14 each illustrate a filtering result with a typical Gaussian filter for a currently-used approach 1.

FIGS. 15 to 20 each illustrate a filtering result with a bilateral filter for a currently-used approach 2.

FIGS. 21 to 26 each illustrate a filtering result with an approach suggested in the present embodiment (with a bilateral filter using a shape image).

### EMBODIMENT

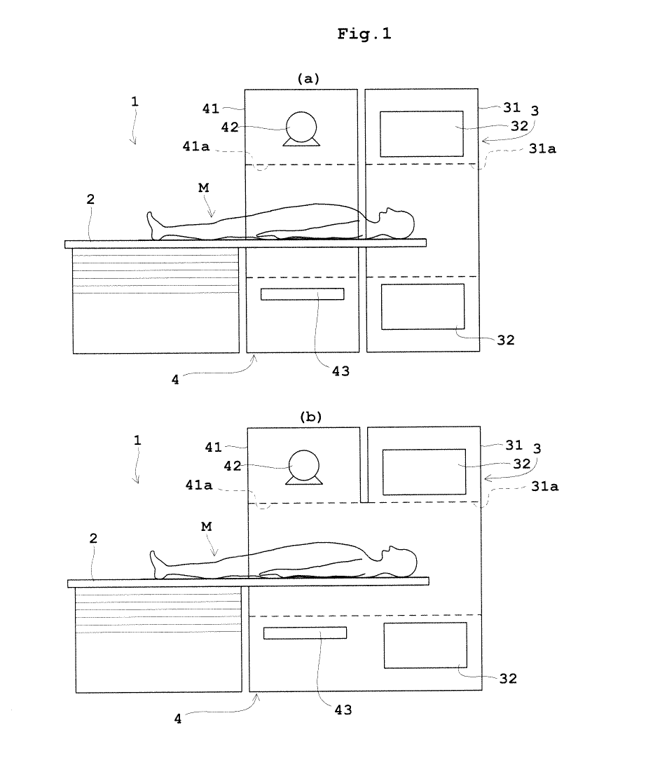

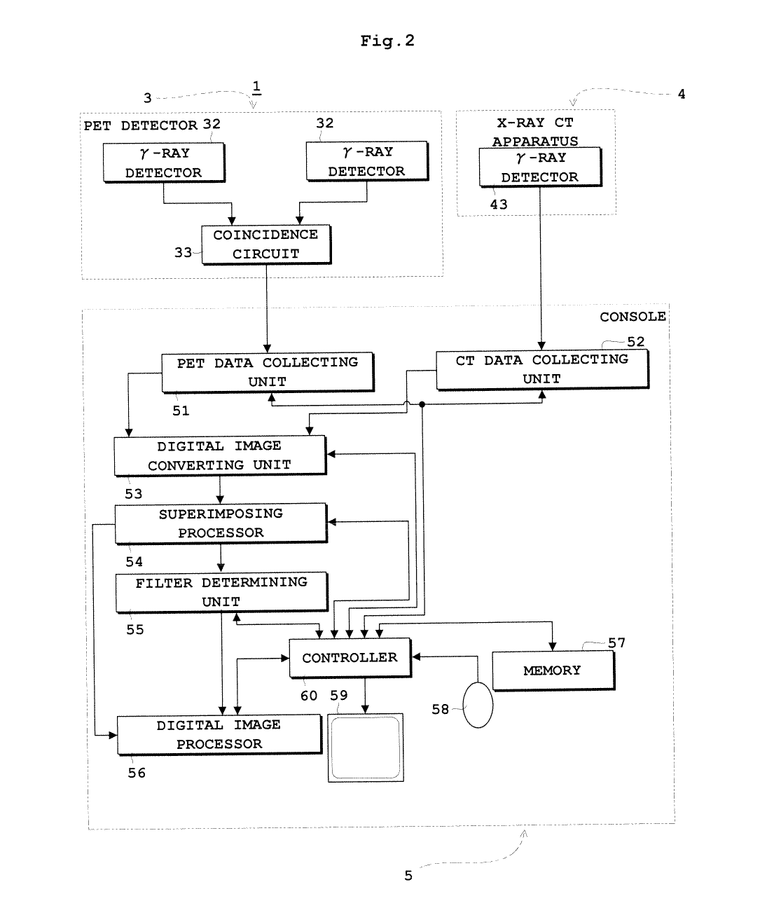

The following describes embodiments of the present invention with reference to drawings. FIG. 1 is a side view of a PET-CT apparatus according to one embodiment of the present invention. FIG. 2 is a block diagram of the PET-CT apparatus according to the embodiment. The present embodiment describes a PET-CT apparatus in combination of a PET apparatus and an X-ray CT apparatus as one example of an imaging apparatus.

As illustrated in FIG. 1, the PET-CT apparatus 1 of the present embodiment includes a top board 2 supporting a subject M placed thereon in a horizontal posture. The top board 2 lifts vertically and moves horizontally along a body axis of the subject M. The PET-CT apparatus 1 includes a PET apparatus 3 conducting diagnosis of the subject M placed on the top board 2. The PET-CT apparatus 1 further includes an X-ray CT apparatus 4 capturing a CT image of the subject M. Here, the PET-CT apparatus 1 corresponds to the imaging apparatus in the present invention.

The PET apparatus 3 includes a gantry 31 with an opening 31a and a γ-ray detector 32 detecting γ-rays generated from the subject M. The γ-ray detector 32 is so arranged as to surround the subject M in a ring shape about the body axis. The γ-ray detector 32 is embedded in the gantry 31. The γ-ray detector 32 includes scintillator blocks 32a, a light guide 32b, and a photomultiplier (PMT) 32c (see FIG. 3). The scintillator block 32a is formed by a plurality of scintillators. The scintillator block 32a converts γ-rays into light, the γ-rays being generated from the subject M with radiopharmaceutical administered thereinto. The light guide 32b guides the converted light. The photomultiplier 32c photoelectrically converts the light to output electric signals. Here, the γ-ray detector 32 and an X-ray detector 43 to be mentioned later correspond to the imaging unit in the present invention. A concrete construction of the γ-ray detector 32 is to be illustrated later in FIG. 3.

The X-ray CT apparatus 4 includes a gantry 41 with an opening 41a. The gantry 41 includes therein an X-ray tube 42 emitting X-rays to the subject M, and an X-ray detector 43 detecting X-rays passing through the subject M. The X-ray tube 42 and the X-ray detector 43 face each other. A motor (not shown) is driven to cause the X-ray tube 42 and the X-ray detector 43 to rotate around the body axis of the subject M in the gantry 41. In the present embodiment, a flat panel X-ray detector (FPD) is adopted as the X-ray detector 43.

In FIG. 1(a), the gantry 31 of the PET apparatus 3 and the gantry 41 of the X-ray CT apparatus 4 are provided individually. Alternatively, as illustrated in FIG. 1(b), both may be constructed integrally.

The following describes a block diagram of the PET-CT apparatus 1. As illustrated in FIG. 2, the PET-CT apparatus 1 includes a console 5 in addition to the top board 2, the PET apparatus 3, and the X-ray CT apparatus 4 mentioned above. The PET apparatus 3 includes a coincidence circuit 33 in addition to the gantry 31 and the γ-ray detector 32 mentioned above.

The console 5 includes a PET data collecting unit 51, a CT data collecting unit 52, a digital image converting unit 53, a superimposing processor 54, a filter determining unit 55, a digital image processor 56, a memory 57, an input unit 58, an output unit 59, and a controller 60. Here, the digital image converting unit 53 corresponds to the digital image converting device in the present invention. The filter determining unit 55 corresponds to the filter determining device in the present invention. The digital image processor 56 corresponds to the digital image processor in the present invention.

The coincidence circuit 33 determines whether or not the γ-ray detector 32 coincidently detects (coincidently counts) γ-rays. The PET data coincidently counted by the coincidence circuit 33 is transmitted to the PET data collecting unit 51 in the console 5. CT data (data X-ray CT) based on the X-rays detected by the X-ray detector 43 is transmitted to the CT data collecting unit 52 in the console 5.

The PET data collecting unit 51 receives and collects the PET data from the coincidence circuit 33 as an analog image (analog image for PET) captured by the PET apparatus 3. The analog image collected by the PET data collecting unit 51 is transmitted to the digital image converting unit 53.

The CT data collecting unit 52 receives and collects the CT data from the X-ray detector 43 as an analog image (analog image for X-ray CT) captured by the X-ray CT apparatus 4. The analog image collected by the CT data collecting unit 52 is transmitted to the digital image converting unit 53.

The digital image converting unit 53 converts the captured image (analog image) into a digital image. In the present embodiment, the digital image converting unit 53 converts the analog image for PET to output a digital image for PET (hereinafter, simply referred to as a “PET image”). Here, the analog image is captured by the PET apparatus 3 and is transmitted via the PET data collecting unit 51. Moreover, the digital image converting unit 53 converts the analog image for X-ray CT to output a digital image for X-ray CT (hereinafter, simply referred to as a “CT image”). Here, the analog image is captured by the X-ray CT apparatus 4 and is transmitted via the CT data collecting unit 52. The digital images (PET image and CT image) are each transmitted to the superimposing processor 54.

The superimposing processor 54 aligns and superimposes the PET image with and on the CT image, the images each being subjected to conversion into the digital image by the digital image converting unit 53. Alternatively, the CT image may act on the PET image as transmission data for absorption correction of the PET image. The PET image and the CT image superimposed by the superimposing processor 54 are transmitted to the filter determining unit 55 and the digital image processor 56.

The filter determining unit 55 determines a filter coefficient in filtering. In the present embodiment, the filter coefficient is determined with the PET image and the CT image. The filter coefficient determined by the filter determining unit 55 is transmitted to the digital image processor 56.

The digital image processor 56 processes the digital image based on the captured image. In the present embodiment, the digital image processor 56 processes the PET image. The PET image is captured by the PET apparatus 3 and is transmitted via the PET data collecting unit 51, the digital image converting unit 53, and the superimposing processor 54. Moreover, the PET image processed by the digital image processor 56 may again be superimposed on the CT image. The CT image is captured by the X-ray CT apparatus 4 and is transmitted via the CT data collecting unit 52, the digital image converting unit 53, and the superimposing processor 54.

The memory 57 writes and stores data on various images and data on the filter coefficient determined by the filter determining unit 55. The various images are collected, converted, or processed by the PET data collecting unit 51, the CT data collecting unit 52, the digital image converting unit 53, the superimposing processor 54, or the digital image processor 56 via the controller 60. The memory 55 reads out the data via the controller 60 to the output unit 59 as appropriate, where the data is outputted. The memory 57 is formed by a storage medium as representative of a ROM (Read-only Memory) and a RAM (Random-Access Memory).

The input unit 58 transmits the data or commands by an operator to the controller 60. The input unit 60 is formed by a pointing device as representative of a mouse, a keyboard, a joystick, a trackball, and a touch panel. The output unit 59 is formed by a display unit as representative of a monitor, and a printer.

The controller 60 controls en bloc each element of the PET-CT apparatus 1 according to the present embodiment. The controller 60 is formed by a central processing unit (CPU) and the like. The controller 60 controls the memory 57 to write and store the data on various images and the data on the filter coefficient determined by the filter determining unit 55 or to transmit the data to the output unit 59, where the data is outputted. The images are collected, converted, or processed by the PET data collecting unit 51, the CT data collecting unit 52, the digital image converting unit 53, the superimposing processor 54, or the digital image processor 56. When the output unit 59 is a display unit, the data is outputted and displayed. When the output unit 59 is a printer, the data is outputted and printed.

The γ-rays generated from the subject M with the radiopharmaceutical administered thereinto are converted into light by a corresponding scintillator block 32a (see FIG. 3) of the γ-ray detector 32, and the converted light is photoelectrically converted by a photomultiplier 32c (see FIG. 3) of the γ-ray detector 32, whereby the light is outputted as electric signals. The electric signals are transmitted as image information (pixel values) to the coincidence circuit 33.

Specifically, when the radiopharmaceutical is administered into the subject M, a positron of a positron-emission type RI (radioactive isotope) annihilates to generate two γ-ray beams. The coincidence circuit 33 checks positions of the scintillator blocks 32a (see FIG. 3) of the γ-ray detector 32 and an incidence timing of the γ-ray beams. Only when the γ-ray beams coincidently enter into the two scintillator blocks 32a facing to each other across the subject M (i.e., the γ-ray beams are coincidently counted), it is determined that information on the transmitted image is valid data. When the γ-ray beams enter into only one of the scintillator blocks 32a, the coincidence circuit 33 considers the γ-ray beams not as the γ-ray beams derived from positron annihilation but as noise, and information on the transmitted image is also determined as noise. Both are invalid.

The image information in the coincidence circuit 33 is transmitted to the PET data collecting unit 51 as the PET data (emission data). The PET data collecting unit 51 collects the transmitted PET data, and transmits the data to the digital image converting unit 53.

The X-ray tube 42 emits X-rays to the subject M while the X-ray tube 42 and the X-ray detector 43 rotate. The X-ray detector 43 converts X-rays emitted externally and passing through the subject M into electric signals, and detects the electric signals. The electric signals converted by the X-ray detector 43 are transmitted to the CT data collecting unit 52 as image information (pixel values). The CT data collecting unit 52 collects a distribution of the transmitted image information as CT data, and transmits the CT data to the digital image converting unit 53.

The digital image converting unit 53 converts an analog pixel value into a digital pixel value. Consequently, the digital image converting unit 53 converts the analog image for PET (PET data) transmitted from the PET data collecting unit 51 into the digital image for PET (PET image), and converts the analog image for X-ray CT (CT data) transmitted from the CT data collecting unit 52 into the digital image for X-ray CT (CT image). Then, the images are transmitted to the superimposing processor 54.

Concrete functions of the subsequent superimposing processor 54, the filter determining unit 55, and the digital image processor 56 are to be described in detail later.

The following describes a concrete construction of the γ-ray detector 32 with reference to FIG. 3. FIG. 3 schematically illustrates a concrete construction of the γ-ray detector.

The γ-ray detector 32 is composed of scintillator blocks 32a, a light guide 32b optically connected to the scintillator blocks 32a, and a photomultiplier 32c optically connected to the light guide 32b. The scintillator blocks each have combination of a plurality of scintillators as detecting elements in a depth direction with different annihilation time. Each of the scintillators in the scintillator block 32a glows due to incident γ-rays and converts it into light, thereby detecting γ-rays. Here, the scintillator block 32a does not always need to have combination of scintillators in the depth direction (r in FIG. 3) with different annihilation times. Moreover, the scintillators are combined in two layers in the depth direction. Alternatively, the scintillator block 32a may be composed of single-layered scintillators.

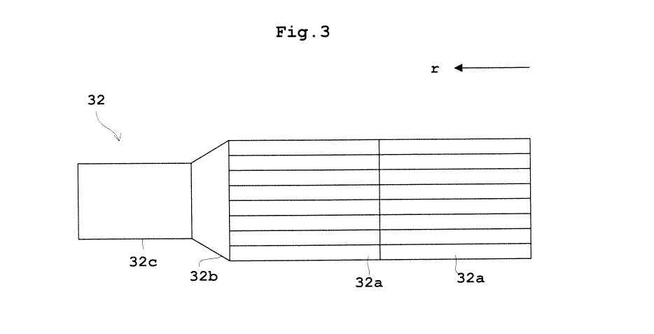

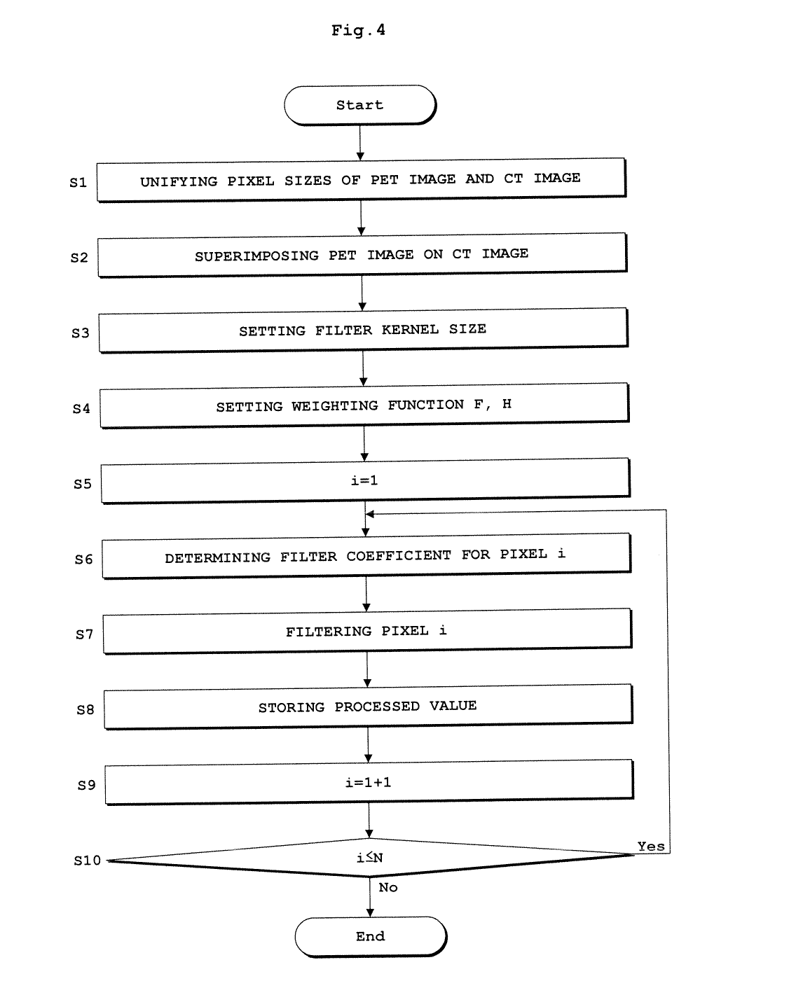

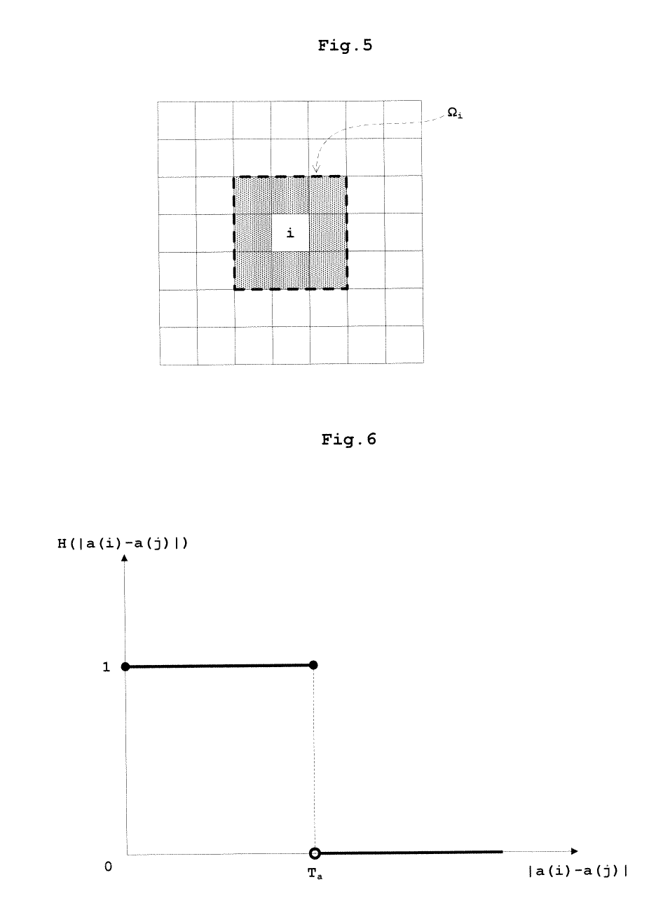

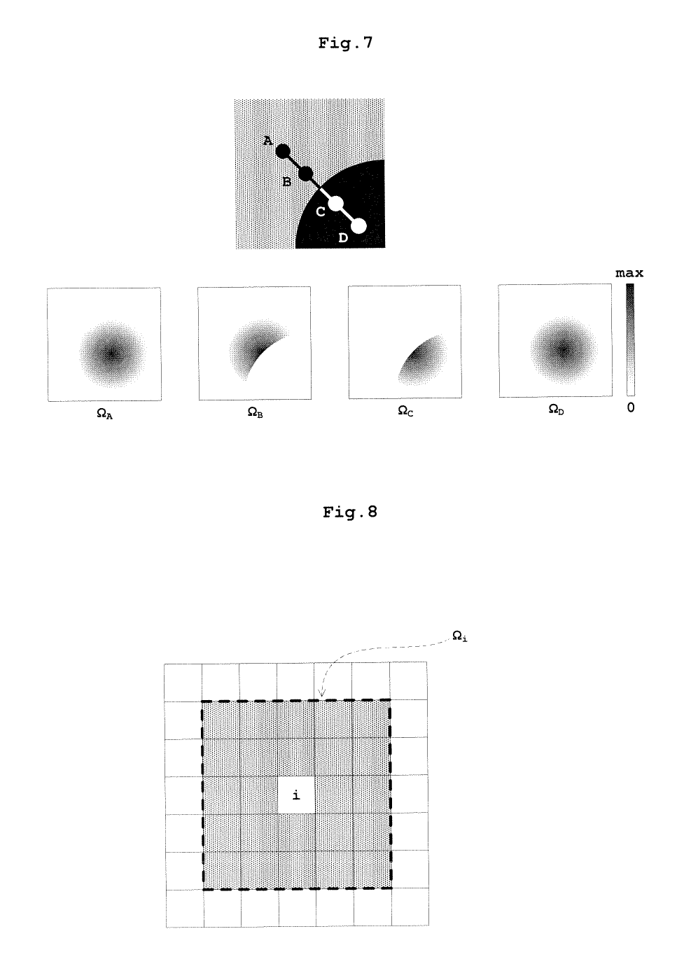

The following describes concrete functions of the superimposing processor 54, the filter determining unit 55, and the digital image processor 56 with reference to FIGS. 4 to 7. FIG. 4 is a flow chart illustrating a series of digital image processing containing filtering. FIG. 5 schematically illustrates a neighboring-pixel set. FIG. 6 illustrates one example of a non-increasing function applying a weighting factor and having a pixel-value difference of the neighboring pixels as a variable. FIG. 7 is a schematic view of a filter kernel.

Here, the digital image to be processed is denoted by A, and another digital image of the same object (a region of interest of the subject M in the present embodiment) as the digital image A to be processed is denoted by B. In the present embodiment, the PET image for the function image is described as one example of the digital image A to be processed, and the CT image for the shape image is described as one example of the other digital image B. Accordingly, noise removal processing (filtering) is performed to the PET image also with the information on the CT image.

(Step S1) Unifying Pixel Sizes of PET Image and CT Image

The CT image typically has a pixel size smaller than that of the PET image. Consequently, the sizes of both the images are unified in advance. In the present embodiment, the pixel size of the CT image is enlarged for unifying the pixel size of the PET image. Here, it should be noted that “enlarging the pixel size” does not mean enlarging one pixel size itself but means the feature that a plurality of pixels in the CT image corresponding to the pixel size of the PET image is unified (combined) into one pixel.

(Step S2) Superimposing PET Image on CT Image

When the PET image is shifted from the CT image, the superimposing processor 54 (see FIG. 2) performs superimposing processing to align and superimpose the PET image with and on the CT image. The following should be noted. That is, the alignment and superimposition does not mean the feature that both the images are displayed on the output unit 59 (see FIG. 2), and the images are manually shifted by the input unit 58 (see FIG. 2). The alignment and superimposition mean that distributions of pixel values of both the images are specified through calculation, and the both image are horizontally shifted or are rotated through calculation so as for the distributions to conform to each other.

(Step S3) Setting Filter Kernel Size

A size of the filter kernel (filter coefficient) (a neighboring-pixel set Ωᵢ) is set for every pixel. In the present embodiment, the filter kernel has a square shape as in FIG. 5. The center pixel of the square filter kernel (to be processed) is a target pixel (see the symbol i in FIG. 5), and pixels around the filter kernel (see gray portions therearound) are neighboring pixels relative to the target pixel, and a set of the neighboring pixels is a neighboring-pixel set (see the symbol Ωᵢ in FIG. 5). In FIG. 5, the filter kernel has a size of nine pixels, i.e., 3 in pixel row by 3 in pixel column, containing the target pixel. Accordingly, the remaining eight neighboring pixels except for the target pixel correspond to adjacent pixels relative to the target pixel.

(Step S4) Setting Weighting Function F, H

The filter determining unit 55 (see FIG. 2) determines the filter coefficient with information on the pixel-to-pixel distance and information on the pixel-value difference between the target pixel to be processed and the neighboring pixel around the target pixel and also with the information on the other digital image (CT image in the present embodiment) B. Specifically, real value functions F and H in the following Equation (3) and (4) are set that influence the characteristic of the filter coefficient.

Here, any given function having a pixel-to-pixel distance as a variable is denoted by E This is a function of applying weight depending on the pixel-to-pixel distance (also referred to as a “weighting function”). In the present embodiment, the symbol F is a Gaussian function of a standard deviation σᵣ. A pixel-to-pixel distance between the neighboring pixel and the target pixel is denoted by r. Assuming that a position vector of the target pixel i from a reference position is r(i), and the position vector of the neighboring pixel j from the reference position is r(j), the r is given by ∥r(i)−r(j)∥. Here, the reference position is not particularly limited. For instance, a pixel is assumed to be an original point, and the original point may be the reference position. Alternatively, the target pixel may always be the reference position. In any cases, in the neighboring-pixel set Ωᵢ in FIG. 5, a pixel-to-pixel distance between the target pixel and the adjacent pixel located upper right, upper left, lower right, or lower left is √2 times the length of a pixel-to-pixel distance between the target pixel and the adjacent pixel located upper, lower, right, or left, respectively. The Gaussian function is a normal distribution. On the other hand, since the symbol r is an absolute value and is always a positive real number, the symbol F is a non-increasing function.

Any given function having a pixel-value difference of the neighboring pixels in another digital image (CT image in the present embodiment) B as a variable is denoted by H. In the present embodiment, the symbol H is a function applying weight depending on edge strength of the CT image B (pixel-value difference between the target pixel and the adjacent pixel). In the present embodiment, the symbol H is a binary function (threshold Tₐ) in FIG. 6. In FIG. 6, a pixel value of the target pixel i in the CT image B for the shape image is denoted by a(i), a pixel value of the neighboring pixel j in the CT image B is denoted by a(j). Accordingly, a pixel-value difference is given by |a(i)−a(j)|. Moreover, the function H having the pixel-value difference |a(i)−a(j)| as a variable is preferably a non-increasing function. For instance, a binary function as illustrated in FIG. 6 is adoptable having a constant function of “1” in an area where the pixel-value difference is equal to or less than the threshold Tₐ, and a constant function of “0” in an area where the pixel-value difference is more than the threshold Tₐ.

In FIG. 6, a function has a constant value of “1” in an area where the pixel-value difference is equal to or less than the threshold Tₐ. However, the constant value a is not limited to “1” as long as a>0 is satisfied. Moreover, two or more thresholds are set (e.g., Tₐ<T_{b}), and a>b>0 is satisfied. Then a multivalued function is adoptable having a constant function value of “a” in an area where a pixel-value difference is equal to or less than the threshold Tₐ, a constant function value of “b” in an area where the pixel-value difference is more than the threshold Tₐ and is equal to or less than the threshold T_{b}, and a constant function value of “0” in an area where the pixel-value difference is more than the threshold T_{b}. Such is adoptable. Moreover, a value of the function does not always need to be constant at a part of the pixel-value difference. For instance, a value of the function may gradually decrease monotonically. Alternatively, a value of the function is constant at a part of the pixel-value difference, whereas a value of the function smoothly decreases monotonically in the other part. Such is adoptable.

(Step S5) i=1

A filter coefficient is calculated and determined for the target pixel i following the Equations (3) and (4) as under (step S6). In addition, filtering is performed to the target pixel i (step S7). Firstly, the target pixel i=1 is set.

(Step S6) Determining Filter Coefficient for Pixel i

Then, if the target pixel i=1 is set in the step S5 or the target pixel i≦N (N is the number of pixels) is set in a step S10 to be mentioned later, a filter coefficient is determined for the target pixel i set to be i+1 (the value of i being incremented by 1 by substituting a right side “i+1” into a left side i) in a step S9 to be mentioned later. Specifically, the filter determining unit 55 (see FIG. 2) determines a filter coefficient W following the Equations (3) and (4) as under in accordance with the information on pixel-value distance between the target pixel i to be processed and the neighboring pixel j around the target pixel (in the present embodiment ∥r(i)−r(j)∥) as well as information on the pixel-to-pixel difference (|a(i)−a(j)|), and also in accordance with the information on the CT image B.

$$\begin{matrix}W ( i , j ) = \frac{w ( i , j )}{{\sum }_{k ∈ {Ω}_{i}} w ( i , k )} & ( 3 ) \\ w ( i , j ) = \frac{F ( ‖ r ( i ) - r ( j ) ‖ )}{{\sum }_{k ∈ {Ω}_{i}} F ( ‖ r ( i ) - r ( k ) ‖ )} \times \frac{H ( \vert a ( i ) - a ( j ) \vert )}{{\sum }_{k ∈ {Ω}_{i}} H ( \vert a ( i ) - a ( k ) ‖ )} & ( 4 )\end{matrix}$$

Where i denotes an index of the target pixel, j denotes an index of the neighboring pixel (adjacent pixel) relative to the target pixel i, w denotes a weighting factor of the neighboring pixel (adjacent pixel) j relative to the target pixel i, Ωᵢ denotes a neighboring-pixel set relative to the target pixel i (see FIG. 5), k denotes a variable belonging to the neighboring-pixel set Ωᵢ, r(i) denotes a position vector of the target pixel i from a reference position, r(j) denotes a position vector of the neighboring pixel j from the reference position, a(i) denotes a pixel value of the target pixel i in the CT image B for a shape image, a(j) denotes a pixel value of the neighboring pixel (adjacent pixel) j in the CT image B, and F and H each denote an any given function (weighting function). As mentioned above, the weighting functions F and H are each preferably a non-increasing function. In the present embodiment, F is a Gaussian function for a standard deviation σᵣ, and H is a binary function as illustrated in FIG. 6. In addition, in the Equation (3), division is made by Σw(i,k) (where Σw(i,k) is a total sum of the variable k(i,k) belonging to the neighboring pixel set Ωᵢ) for normalization of the filter coefficient W.

That is, the present embodiment relates to an approach of edge-preserved smoothing filtering to a nuclear medicine image (a PET image A in the present embodiment) using information on an organ contour in a CT image B for a shape image as prior information. The pixel value of the nuclear medicine image (PET image A) has physiological information mentioned above, and thus reflects an organ function (e.g., metabolic capacity and blood flow). Accordingly, functions are various between organs. In other words, it is considered that the pixel value differs depending on organs. Then, a filter coefficient W of the smoothing filter to the nuclear medicine image (PET image A) is calculated from the above Equations (3) and (4) using pixel value information (|a(i)−a(j)| in the present embodiment) of the shape image (CT image Bin the present embodiment).

In the currently-used bilateral filter, it is determined whether or not an edge exists between pixels in the nuclear medicine image (PET image A) using the pixel value information of the nuclear medicine image (PET image A) itself. Consequently, a false edge derived from noises is likely to be detected and preserved. Then, in the present embodiment, it is determined whether or not an edge exists between pixels in the nuclear medicine image (PET image A) using the pixel-value information of another digital image, i.e., a shape image (CT image B) with high resolution and low noise. As a result, smoothing with an edge-preserved smoothing filter is obtainable with no influence of a noise level of the nuclear medicine image (PET image A).

For instance, a binary function is used for a function H for determining a size of the edge in the shape image (CT image) B as illustrated in FIG. 6. The binary function has a value of “0” (with edge) when a pixel-value difference |a(i)−a(j)| is more than a threshold Tₐ, and has a value of “1” (with no edge) when the difference is equal to or less than the threshold Tₐ. Using such a binary function causes smoothing only within an area not across the edge as illustrated in FIG. 7. This achieves noise reduction as well as preserve of the edge (space resolution).

As illustrated on the upper of FIG. 7, when a target pixel is A and D in the CT image, the target pixel is sufficiently spaced away from the edge, and thus the filter kernel has a size (neighboring-pixel set Ω_{A}, Ω_{D}) not overlapping the edge. Consequently, as illustrated on the lower left end and lower right end of FIG. 7, smoothing is performed by normal weighting using the function H having a value of “1” (with no edge).

In contrast to this, when a target pixel is B and C in the CT image as illustrated on the upper of FIG. 7, the target pixel is close to the edge, and thus the filter kernel has a size (neighboring-pixel set ω_{B}, ω_{C}) overlapping the edge. Consequently, as illustrated on the second from the left and from the right of the lower of FIG. 7, a weighting function H having a value of “1” (with no edge) is used in the area not overlapping the edge, and a weighting function H having a value of “0” (with edge) is used in the area overlapping the edge. As a result, smoothing is performed only to the area not overlapping the edge by normal weighting and to the area overlapping the edge by smaller weighting (“0” in FIGS. 6 and 7).

At this time, the image information to be referred to is not the currently-used nuclear medicine image itself but a shape image (CT image) with high resolution and low noise. This achieves smoothing with no influence of a false edge in the nuclear medicine image derived from noise.

(Step S7) Filtering Pixel i

After determination of the filter coefficient W from the above Equations (3) and (4) in the step S6, the digital image processor 56 (see FIG. 2) processes the digital image (PET image in the present embodiment) A to be processed using the filter coefficient W determined by the filter determining unit 55 (see FIG. 2). In this way, filtering to the target pixel i (calculation of a weighted average value) is performed.

(Step S8) Storing Processed Value

The value after the filtering is written and stored into another memory region of the memory 57 (see FIG. 1) different from a memory region for the pre-processing PET image A (i.e., an original image). Such storage achieves individual storage of the processed image and the pre-processing PET image A (original image) without overwriting the pre-processing PET image A (original image).

(Step S9) i=i+1

Setting of i=i+1 causes the value of i to be incremented by 1. Here, the symbol “=” does not mean an equal sign but means substitution. Accordingly, substitution of a right side “i+1” for a left side causes increment of the value i by 1.

(Step S10) i≦N

The number of pixels is denoted by N. Then it is determined whether or not i is equal to or less than N. If i equals N, it is determined that filtering for all the pixels is not completed. Then the process returns to the step S6, and the steps S6 to S10 are performed continuously. Consequently, the steps S6 to S10 are repeatedly performed until the filtering to all the pixels is completed. If i is larger than N, it is determined that the filtering to all the pixels is completed. Then a series of digital image processing in FIG. 4 is completed.

With the digital image processing method according to the present embodiment, the filter coefficient W is determined also with the other digital image (CT image in the present embodiment) B. This achieves the filtering with no influence of a noise level in the digital image (the PET image in the present embodiment) A to be processed. As a result, maintenance of the space resolution as well as noise reduction is both obtainable.

The other digital image B mentioned above is preferably a shape image as representative of the CT image B as in the present embodiment. Especially, when the image to be processed is a digital image based on nuclear medicine data (nuclear medicine image), the nuclear medicine image has physiological information, and thus is referred to as a “function image”. On the other hand, the nuclear medicine image lacks anatomical information. Consequently, use of the shape image with anatomical information as the other digital image (CT image) B leads to use of the shape image (CT image B in the present embodiment) with a high space resolution and low noise. This produces a more sufficient effect.

Moreover, the function (a weighting function H in the present embodiment) for determining the filter coefficient W having a variable for the pixel-value difference (|a(i)−a(j)| in the present embodiment) is preferably a non-increasing function. When the pixel-value difference is small, smoothing is performable with a large function. When the pixel-value difference is large, an edge with the large difference can be preserved with a small function. Here, the “non-increasing function” is applicable as long as the function does not increase with increase of the pixel-value difference. Accordingly, a value of the function may be constant at a portion of the pixel-value difference. Consequently, as illustrated in FIG. 6, a portion of the pixel-value difference equal to or less than a threshold (Tₐ in FIG. 6) has a constant value “a” (where a>0) (a=1 in FIG. 6), whereas a portion of the pixel-value difference more than the threshold (Tₐ in FIG. 6) has a constant function “0”. A function composed of such constant values is a non-increasing function.

Moreover, the Equations (3) and (4) are generalized to yield the following equation. That is, the filter coefficient W(i,j) of the digital image A to be processed for the filter processing is given by the equations:

W(i,j)=w(i,j)/Σw(i,k), wherein Σw(i,k) is a total sum of w(i,k) of the variable k belonging to the neighboring-pixel set Ωᵢ, and

w(i,j)=F(∥r(i)−r(j)∥)×H(|I_{b}(i)−I_{b}(j)|)

The above Equations (1) to (4) include common symbols. However, I_{b}(i) denotes a pixel value of a target pixel i in the other digital image B, and I_{b}(j) denotes a pixel value of a neighboring pixel j in the other digital image B. Moreover, F(∥r(i)−r(j)∥)/ΣF(∥r(i)−r(k)∥) in Equation (4) (where, ΣF(∥r(i)−r(k)∥ is a total sum of F(∥r(i)−r(k)∥) as a variable k belonging to the neighboring pixel set Ωᵢ) is generalized to F(∥r(i)−r(j)∥) in the equation in the previous paragraph. Similarly, H(|a(i)−a(j)|)/ΣH(|a(i)−a(k)|) in the Equation (4) (where, ΣH(|a(i)−a(k)| is a total sum of H(|a(i)−a(k)| as a variable k belonging to the neighboring-pixel set Ωᵢ) is generalized to H(|I_{b}(i)−I_{b}(j)|) in the equation in the previous paragraph.

From the above equations, a weighting factor w(i,j) of the neighboring pixel j relative to the target pixel i is determined with the any given function H having the pixel-value difference from the neighboring pixel in the other digital image (CT image in the present embodiment) B as the variable, and additionally the filter coefficient W(i,j) is determined with the weighting factor w(i,j). Consequently, the filter coefficient W(i,j) is determined also with the other digital image (CT image) B.

The PET-CT apparatus 1 according to the present embodiment having the above construction includes the filter determining unit 55 determining the filter coefficient in filtering, and the digital image processor 56 processing the digital image (PET image A in the present embodiment) based on the captured image. The filter determining unit 55 determines the filter coefficient W with the information on the pixel-to-pixel distance and the information the pixel-value difference between the target pixel to be processed and the neighboring pixel and also with the information on the other digital image (CT image in the present embodiment) B. The digital image processor 56 processes the digital image (PET image) A to be processed with the filter coefficient W determined by the filter determining unit 55. As noted above, the filter coefficient is determined also with the other digital image (CT image) B. This achieves filtering with no influence of a noise level in the digital image (PET image) A to be processed. Consequently, as described in the digital image processing method according to the present embodiment, maintenance of the space resolution and noise reduction is both obtainable.

The PET-CT apparatus 1 according to the present embodiment further includes an imaging unit (γ-ray detector 32 or X-ray detector 43 in the present embodiment) with a camera function for imaging a static image or a video function for imaging a moving video picture, and a digital image converting unit 53 converting the image by the γ-ray detector 32 into a digital image. Such is preferable. With the imaging unit (γ-ray detector 32 or X-ray detector 43) and the digital image converting unit 53, the imaging unit allows imaging of the static image or the moving video picture while the digital image converting unit 53 allows conversion of the image (analog image) by the imaging unit (γ-ray detector 32 or X-ray detector 43) into the digital image (PET image or CT image in the present embodiment), and the digital image processor 56 allows processing of the converted digital image (CT image in the present embodiment).

The present embodiment describes the nuclear medicine diagnosis apparatus for conducting nuclear medicine diagnosis as one example of the imaging apparatus, and describes the PET-CT apparatus 1 in combination of the PET apparatus and the X-ray CT apparatus as one example of the nuclear medicine diagnosis apparatus. The digital image processor 56 preferably processes the digital image (PET image in the present embodiment) based on the nuclear medicine data obtained through the nuclear medicine diagnosis. As also described in the digital image processing method according to the embodiment, the digital image (nuclear medicine image) based on the nuclear medicine data obtained through the nuclear medicine diagnosis is a function image, and thus lacks anatomical information. Accordingly, it is assumed that the digital image (PET image in the present embodiment) A to be processed corresponds to a digital image in accordance with nuclear medicine data, and another digital image B (CT image in the present embodiment) corresponds to a shape image. Consequently, use of the shape image with anatomical information as the other digital image (CT image) B leads to use of a shape image (CT image B) with a high space resolution and low noise. As a result, even when the digital image (PET image A) to be processed is a nuclear medicine image as a function image lacking anatomical information, both maintenance of the space resolution and noise reduction is obtainable.

[Experimental Result]

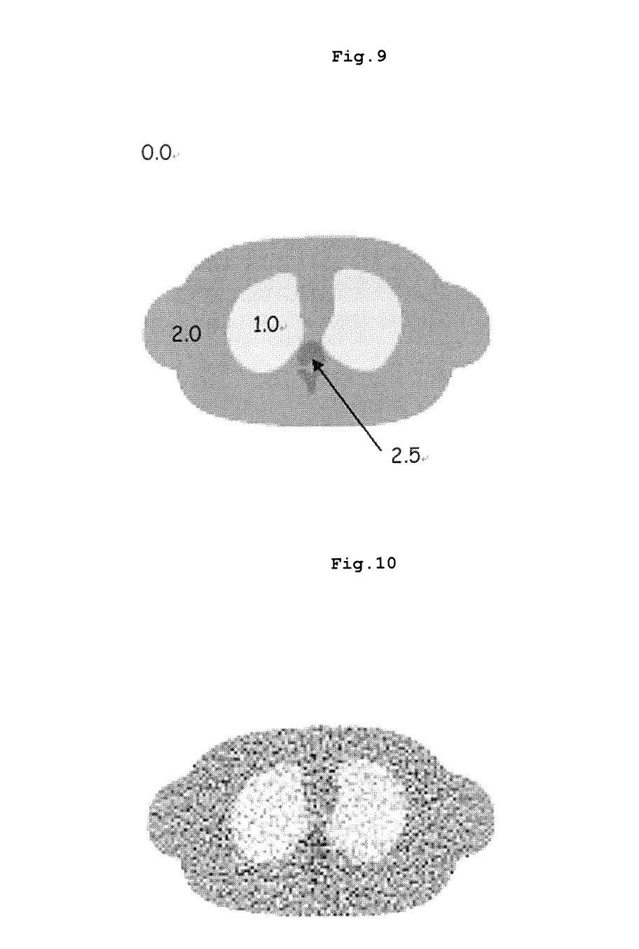

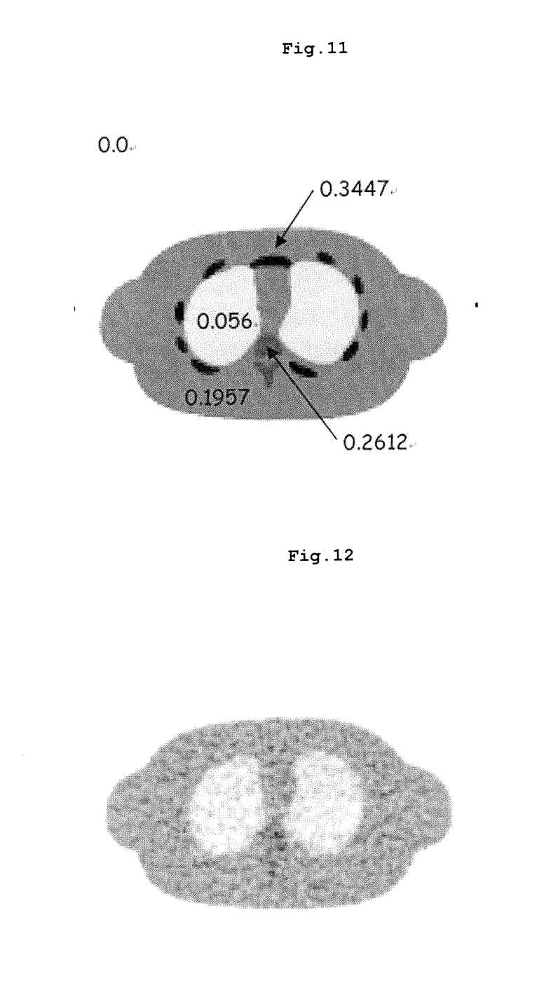

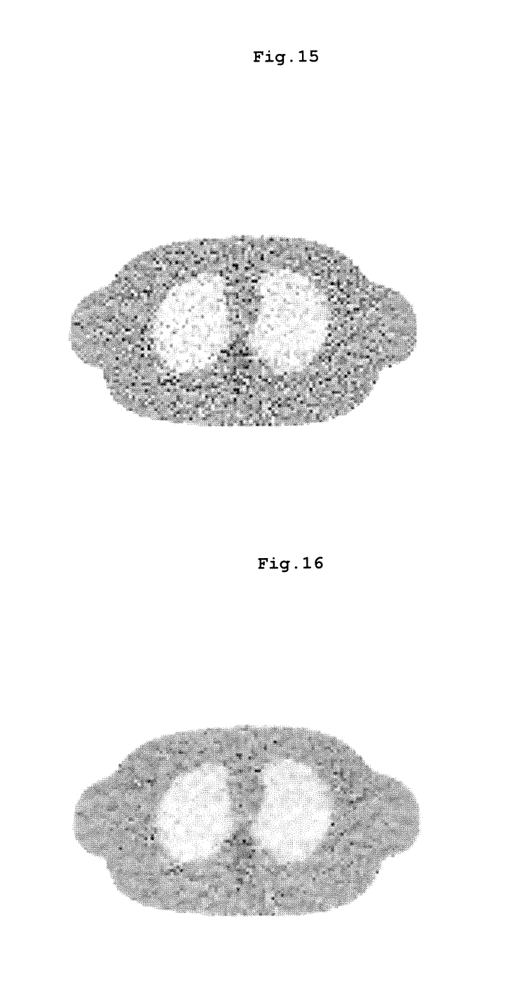

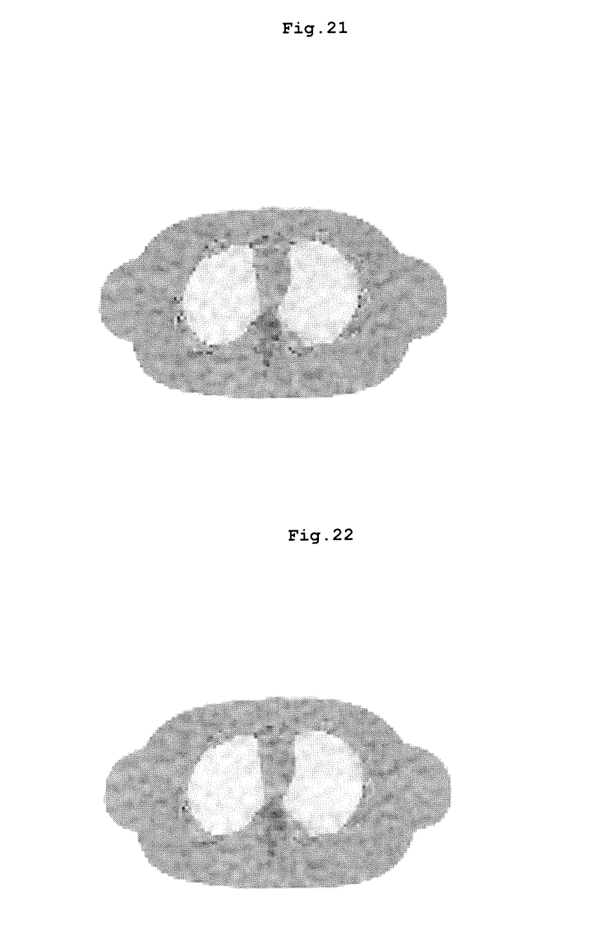

The following describes experimental results of simulation experiments with reference to FIGS. 9 to 26. FIG. 9 is an original image used for demonstration data (simulation experiments). FIG. 10 is an image with noise artifactually applied to the original image of FIG. 9. FIG. 11 is a shape image used for demonstration data (simulation experiments). Moreover, experimental results of the currently-used approaches 1 and 2 are also illustrated for comparison of the suggested approach in the present embodiment (i.e., a bilateral filter with a shape image). FIGS. 12 to 14 are filtering results each by a typical Gaussian filter for the currently-used approach 1. FIGS. 15 to 20 are filtering results each by a bilateral filter for the currently-used approach 2. FIGS. 21 to 26 are filtering results of the suggested approach in the present embodiment (i.e., a bilateral filter with the shape image).

For a simulation experiment, noise is artifactually applied to an original image in FIG. 9 to generate an image with noise (see FIG. 10). Then filtering is performed by the currently-used approaches 1 and 2 and the suggested approach in the present embodiment. The suggested approach in the present embodiment adopts a shape image in FIG. 11 for the filtering. The images each have a pixel size of 128 by 128. FIGS. 9 and 11 include numeric values each indicating a pixel value representative of a region. Here, smoothing parameters σᵣ and σₓ are half-value widths. Accordingly, the smoothing parameters σᵣ and σₓ are each to be referred to as a “half-value width”.

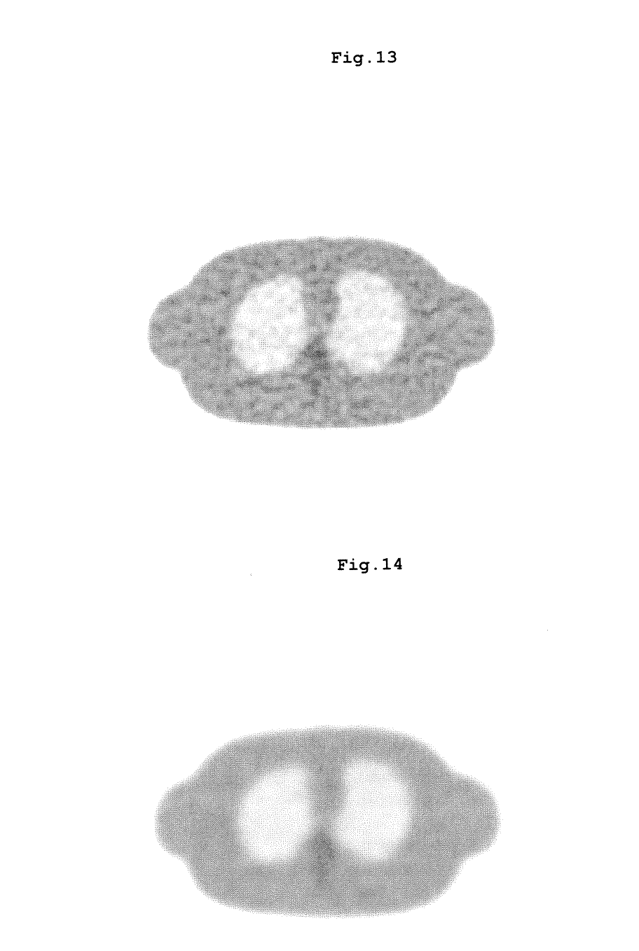

For the currently-used approach 1, FIGS. 12 to 14 illustrate filtering results each by a typical Gaussian filter (half-value width σᵣ=1.5, 2.0, 4.0 pixels). FIG. 12 illustrates the result with a half-value width σᵣ of 1.5 pixels. FIG. 13 illustrates the result with a half-value width σᵣ of 2.0 pixels. FIG. 14 illustrates the result with a half-value width σᵣ of 4.0 pixels.

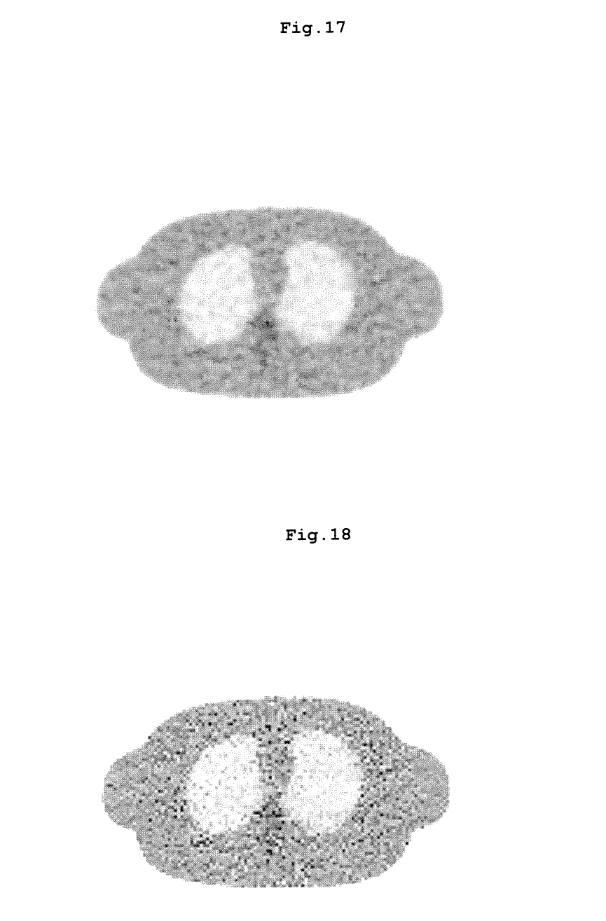

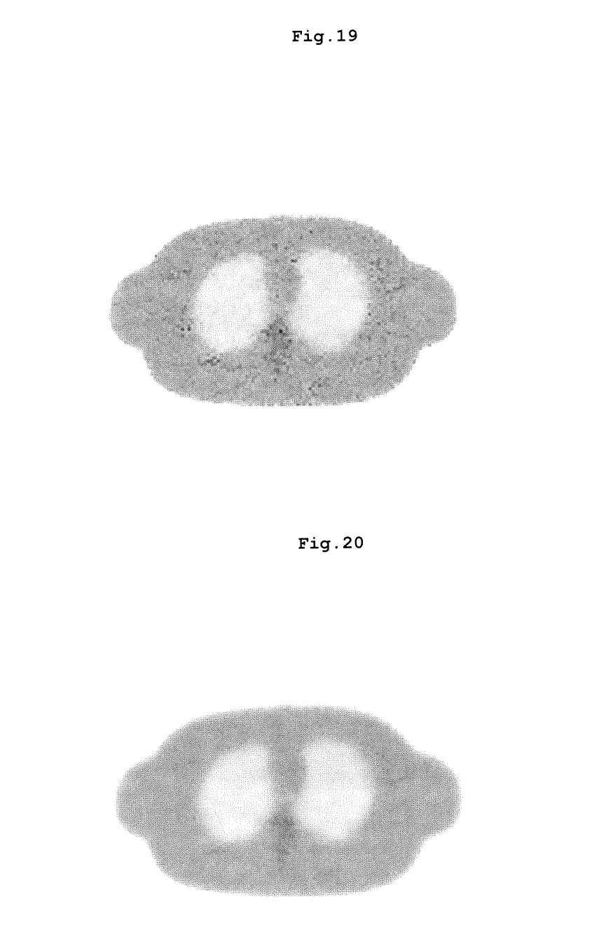

For the currently-used approach 2, FIGS. 15 to 20 each illustrates a filtering result with a bilateral filter. In the bilateral filter, weighting depending on a pixel-to-pixel distance is a Gaussian function with a half-value width σᵣ of 2.0 and 4.0 pixels, and weighting depending on a pixel-value difference is a Gaussian function with a half-value width σₓ of 1.0, 3.0, and 6.0. Consequently, a combination result of the half-value widths σᵣ and σₓ has six patterns in total which are illustrated in FIGS. 15 to 20. FIG. 15 illustrates the result with a half-value width σᵣ of 2.0 pixels and a half-value width σₓ of 1.0. FIG. 16 illustrates the result with a half-value width σᵣ of 2.0 pixels and a half-value width σₓ of 3.0. FIG. 17 illustrates the result with a half-value width σᵣ of 2.0 pixels and a half-value width σₓ of 6.0. Moreover, FIG. 18 illustrates the result with a half-value width σᵣ of 4.0 pixels and a half-value width σₓ of 1.0. FIG. 19 illustrates the result with a half-value width σᵣ of 4.0 pixels and a half-value width σₓ of 3.0. FIG. 20 illustrates the result with a half-value width σᵣ of 4.0 pixels and a half-value width σₓ of 6.0.

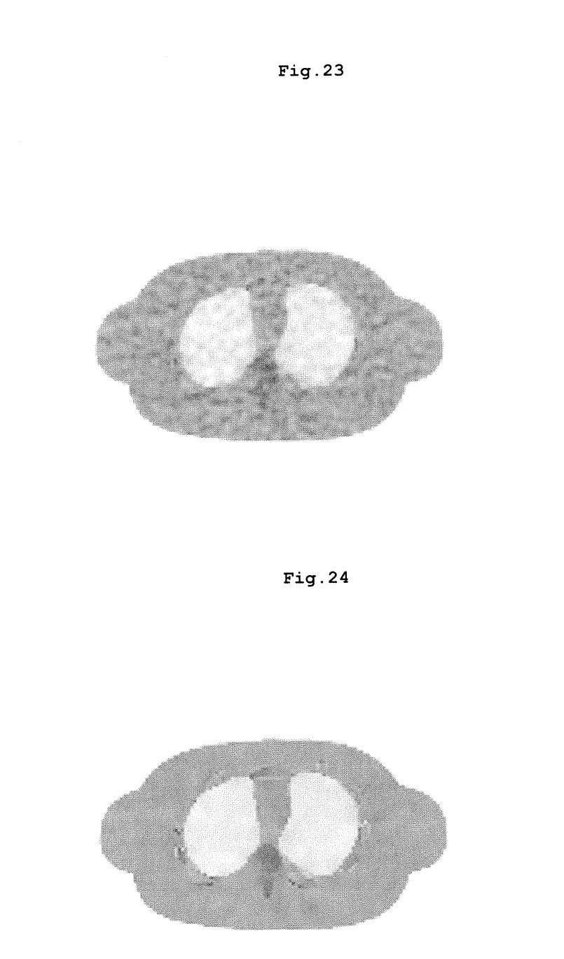

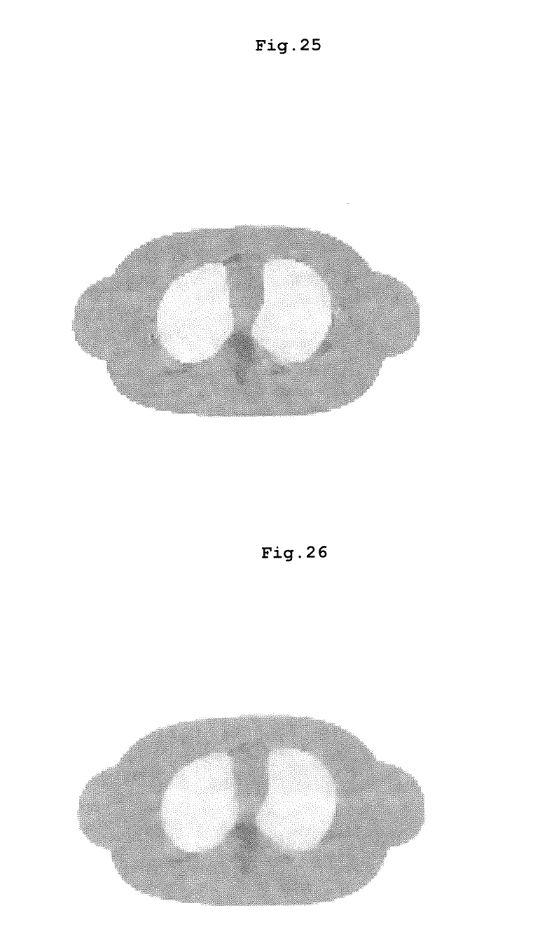

Finally, FIGS. 21 to 26 each illustrate a filtering result with the suggested approach in the present embodiment (a bilateral filter with a shape image). In the suggested approach, weighting depending on a pixel-to-pixel distance is a Gaussian function with a half-value width σᵣ of 2.0 and 4.0 pixels and weighting depending on a pixel-value difference is a Gaussian function with a half-value width σₓ of 0.05, 0.1, and 0.2. Consequently, a combination result of the half-value width σᵣ and σₓ has six patterns in total which are illustrated in FIGS. 21 to 26. FIG. 21 illustrates the result with a half-value width σᵣ of 2.0 pixels and a half-value width σₓ of 0.05. FIG. 22 illustrates the result with a half-value width σᵣ of 2.0 pixels and a half-value width σₓ of 0.1. FIG. 23 illustrates the result with a half-value width σᵣ of 2.0 pixels and a half-value width σₓ of 0.2. FIG. 24 illustrates the result with a half-value width σᵣ of 4.0 pixels and a half-value width σₓ of 0.05. FIG. 25 illustrates the result with a half-value width σᵣ of 4.0 pixels and a half-value width σₓ of 0.1. FIG. 26 illustrates the result with a half-value width σᵣ of 4.0 pixels and a half-value width σₓ of 0.2.

It is determined from the results of FIGS. 12 to 14 that, in the currently-used Gaussian filter, noise is reduced with increase in width of the Gaussian function (half-value width σᵣ) having a pixel-to-pixel distance as a variable increases. On the other hand, a contour blurs and thus a space resolution is reduced. Moreover, it is determined from the results of FIGS. 15 to 20 that, in the currently-used bilateral filter, noise cannot be reduced satisfactorily (see FIGS. 15, 16, and 18) or noise is reduced but a contour blurs (see FIGS. 17, 19, and 20) even with control of a width of the Gaussian function (half-value width σᵣ) having a pixel-to-pixel distance as a variable and a width of the Gaussian function (half-value width σₓ) depending on a pixel-value difference. Consequently, maintenance of the space resolution and noise reduction is incompatible.

In contrast to this, it is determined from the results of FIGS. 21 to 26 that, in the suggested approach, maintenance of the space resolution and noise reduction are compatible by individual control of the width of the Gaussian function (half-value width σᵣ) having a pixel-to-pixel distance as a variable and a width of the Gaussian function (half-value width σₓ) depending on a pixel-value difference of the shape image.

Therefore, it is found from the above experimental results that the suggested filtering with the bilateral filter using the shape image is more effective than the currently-used filtering (i.e., filtering with a typical Gaussian filter or filtering with a bilateral filter using not a shape image but an original image itself).

The present invention is not limited to the above embodiments, but may be modified as under.

(1) In the embodiments mentioned above, a PET-CT apparatus in combination of a PET apparatus and an X-ray CT apparatus is described as one example. Alternatively, a general medical image apparatus (e.g., a CT apparatus, an MRI apparatus, an ultrasonic tomography apparatus, and a tomography apparatus for nuclear medicine), a nondestructive testing CT apparatus, a digital camera, and a digital video camera is applicable singly or in combination.

(2) In the embodiments mentioned above, a PET-CT apparatus in combination of a PET apparatus and an X-ray CT apparatus is described as one example of the imaging apparatus. Alternatively, a PET apparatus is applicable singly. For instance, a CT image captured by an external X-ray CT apparatus is transferred to a PET apparatus to determine a filter coefficient with the transferred CT image. Such is adoptable. Similarly, a nuclear medicine diagnosis apparatus other than a PET apparatus (e.g., a SPECT apparatus) is applicable singly, and a filter coefficient may be determined with another digital image (e.g., CT image) captured by an external apparatus.

(3) The embodiments describe filtering of the PET image with the CT image. This is not limited to the PET-CT apparatus. Combination of an X-ray CT apparatus and a SPCT apparatus for filtering an SPECT image with a CT image, combination of an MRI apparatus and a PET apparatus for filtering a PET image with an MRI image, and combination of an MRI apparatus and an SPCT apparatus for filtering an SPECT image with an MRI image are also applicable. In this case, the PET image or the SPECT image is used for a nuclear medicine image, whereas the CT image or the MRI image is used for a shape image.

(4) In the embodiments mentioned above, a multimodality apparatus such as a PET-CT apparatus in combination of a PET apparatus and an X-ray CT apparatus is described as one example of the imaging apparatus. Alternatively, an MRI apparatus is applicable singly. For instance, a T1 highlighted image and a diffusion highlighted image are each generated from an MRI image captured by an MRI apparatus. Here, the diffusion highlighted image is assumed as a digital image A to be processed, and the T1 highlighted image is assumed as another digital image B. Then, a filter coefficient is determined with the T1 highlighted image, and the diffusion highlighted image is processed with the filter coefficient. Such is adoptable. As noted above, two image captured in the same apparatus is usable.

(5) In the embodiment mentioned above, the filter kernel in FIG. 5 has a square shape of 3 pixels in row by 3 pixels in column However, another size is adoptable. For instance, as illustrated in FIG. 8, a square shape of 5 pixels in row by 5 pixels in column is adoptable. With the square shape of 3 pixels in row by 3 pixels in column as illustrated in FIG. 5, pixels other than the target pixel belonging to the neighboring pixel set Ωᵢ are all adjacent pixels. In contrast to this, with the square shape of 5 pixels in row by 5 pixels in column in FIG. 8, pixels belonging to the neighboring pixel set Ωᵢ other than the pixel in an adjacent image are contained in neighboring pixels.

(6) In the embodiments mentioned above, the filter kernel is square. However, the filter kernel is not particularly limited, and may be in a rectangle or polygon shape.

(7) In the embodiments mentioned above, if one filter kernel is set, the same filter kernel is used for performing the step S6 to S10 repeatedly until filtering to all pixels is completed as illustrated in the flow chart of FIG. 4. Alternatively, a process is returned from the step S10 to the step S3 to update a new a filter kernel for every increment of a value of the target pixel i by one.

(8) In the embodiments mentioned above, a weighting function F having a pixel-to-pixel distance as a variable is a Gaussian function. Alternatively, any function other than the Gaussian function is adoptable. However, a non-increasing function is preferable. In addition, a binary function or a multivalued function such as the weighting function H in the embodiments may be adopted.

(9) In the embodiments mentioned above, the weighting function H having a pixel-value difference as a variable is a binary function. Alternatively, any function other than the binary function is adoptable. However, smoothing is performable using a function with a large value when the pixel-value difference is small, and preserve of an edge with a large difference value is obtainable by a function with a small value when the pixel-value difference is large. Taking it into consideration, a non-increasing function is preferable. Alternatively, as mentioned above, a multivalued function is applicable. Moreover, a Gaussian function as the weighting function F in the embodiments is applicable. That is, a value of the function may be reduced monotonically.

### INDUSTRIAL APPLICABILITY

As noted above, the present invention is suitable for a general medical image apparatus (e.g., a CT apparatus, an MRI apparatus, an ultrasonic tomography apparatus, and a tomography apparatus for nuclear medicine), a nondestructive testing CT apparatus, a digital camera, and a digital video camera.

### REFERENCE SIGN LIST

1 . . . PET-CT apparatus

32 . . . γ-ray detector

43 . . . X-ray detector

53 . . . digital image converting unit

55 . . . filter determining unit

56 . . . digital image processor

A . . . digital image to be processed (PET image)

B . . . another digital image (CT image)

1. A digital image processing method for determining a filter coefficient in accordance with information on a pixel-to-pixel distance and information on a pixel-value difference between a target pixel to be processed and a neighboring pixel around the target pixel, and processing a digital image with the determined filter coefficient, the method comprising:
when a digital image to be processed is denoted by A and another digital image B of the same object as the digital image A is denoted by B, determining the filter coefficient to process the digital image A also with information on the other digital image B.

2. The digital image processing method according to claim 1, wherein
the other digital image B is a shape image.

3. The digital image processing method according to claim 1, wherein
a function having a pixel-value difference as a variable for determining the filter coefficient is a non-increasing function.

4. The digital image processing method according to claim 1, wherein
when an index of the target pixel is denoted by i, an index of the neighboring pixel relative to the target pixel i is denoted by j, a weighting factor of the neighboring pixel j relative to the target pixel i is denoted by w, a neighboring-pixel set around the target pixel i is denoted by Ωᵢ, a variable belonging to the neighboring-pixel set Ωᵢ is denoted by k, a position vector of the target pixel i from a reference position is denoted by r(i), a position vector of the neighboring pixel j from the reference position is denoted by r(j), a pixel value of a target pixel i in the other digital image B is denoted by I_{b}(i), a pixel value of the neighboring pixel j in the other digital image B is denoted by I_{b}(j), an any given function having a pixel-to-pixel distance as a variable is denoted by F, and an any given function having a pixel-value difference from the neighboring pixel in the other digital image B as a variable is denoted by H, the filter coefficient W(i,j) of the digital image A to be processed for filtering is given by equations:
W(i,j)=w(i,j)/Σw(i,k),
where Σw(i,k) is a total sum of w(i,k) of the variable k belonging to the neighboring-pixel set Ωᵢ, and
w(i,j)=F(∥r(i)−r(j)∥)×H(|I_{b}(i)−I_{b}(j)|).

5. An imaging apparatus, comprising:
a filter determining device determining a filter coefficient in filtering; and
a digital image processor processing a digital image in accordance with a captured image,
when a digital image to be processed is denoted by A and another digital image B of the same object as the digital image A is denoted by B, the filter determining device determining the filter coefficient with information on a pixel-to-pixel distance and information on a pixel-value difference between a target pixel to be processed and a neighboring pixel around the target pixel and also with information on the other digital image B, and the digital image processor processing the digital image A to be processed with the filter coefficient determined by the filter determining device.

6. The imaging apparatus according to claim 5, further comprising:
an imaging unit with a camera function for imaging a static image or a video function for imaging a moving video picture; and
a digital image converting device converting the image captured by the imaging unit into a digital image.

7. The imaging apparatus according to claim 5, wherein
the imaging apparatus is a nuclear medicine diagnosis apparatus conducting nuclear medicine diagnosis, and
the digital image processor processes a digital image in accordance with nuclear medicine data obtained from the nuclear medicine diagnosis.

8. The imaging apparatus according to claim 7, wherein
the digital image A to be processed corresponds to a digital image in accordance with the nuclear medicine data, and the other digital image B corresponds to a shape image.
